(19) United States
(12) Patent Application Publication
(10) Pub. No.: US 2005/0287124 A1
(43) Pub. Date: Dec. 29, 2005

(54) Method for inducing bone marrow-derived cells in injured epithelial site and a method for repairing injured epithelium

(75) Inventor: Mamoru Watanabe, Tokyo (JP)

Correspondence Address: GREENBLUM & BERNSTEIN, P.L.C., 1950 ROLAND CLARKE PLACE, RESTON, VA, 20191, US

(73) Assignee: ASAHI KASEI MEDICAL CO., LTD., Tokyo (JP)

(21) Appl. No.: 11/092,763

(22) Filed: Mar. 30, 2005

(30) Foreign Application Priority Data
Mar. 31, 2004 (JP) 2004-103840

(51) Int. Cl. A61K 45/00; C12N 5/08

(52) U.S. Cl. 424/93.7; 435/372

(57) ABSTRACT

It is an object of the present invention to provide a method for repairing injured epithelial cells, impossibility of the repair of which results in fatal consequences. The present invention provides a method for inducing bone marrow-derived cells into the injured epithelial site of a patient and a method for repairing an injured epithelium, each of which comprises removing leukocytes from the blood that has been collected from the patient with injured epithelial cells, and then retransfusing the remaining blood to said patient.

### TECHNICAL FIELD

The present invention relates to a method for inducing bone marrow-derived cells into an injured epithelial site and a method for repairing an injured epithelium. Specifically, the present invention relates to a method for repairing an injured epithelium by inducing bone marrow-derived cells into epithelial cells that constitute an inner wall such as the intestinal tract in a human body. More specifically, the present invention relates to a method for repairing an injured epithelium by passing the blood collected from a patient with injured epithelial cells through a blood treatment means having a means for removing leukocytes from the blood and retransfusing the remaining blood to the above patient, so as to induce bone marrow-derived cells into the injured epithelial site.

### BACKGROUND ART

Renovation of the intestinal epithelium is continuously carried out in all the life of a living body. It is said that such renovation is repeated at intervals of several days. It is considered that the intestinal epithelium is regenerated and renovated at a minimum unit consisting of a villus projecting from the alimentary canal and an intestinal crypt that is a cavity adjacent thereto. Intestinal epithelial stem cells existing at the bottom of the intestinal crypt repeatedly self-replicate. At the same time, such intestinal epithelial stem cells supply intestinal epithelial precursor cells, which actively proliferate and are capable of differentiating into main 4 types of cells (entericytes, goblet cells, enteroendcrine cells, and Paneth cells) that constitute the intestinal epithelium. While these cells remove from the intestinal crypt to the direction of a villus, they become mature epithelial cells. When the mature epithelial cells reach the edges of the villi, they become dead cells and drop into the lumen.

It is generally recognized that the intestinal epithelium is originally a tissue with the highest regenerative ability. However, in serious radiation injuries or certain intractable inflammatory bowel diseases, if the repair of the mucosal epithelium becomes impossible, it results in fatal consequences.

Accordingly, clarification of the origin of intestinal epithelial cells, characteristics of molecules that are specifically expressed, and a mechanism of controlling growth or differentiation thereof, are important for the development of a novel method for treating the aforementioned diseases. Regarding these factors, a publication suggests the possibility of the presence of an epithelial cell-supplying mechanism other than the intestinal tract (Journal of Gastroenterology, the Japanese Society of Gastroenterology, Vol. 100, No. 12, pp. 13-18). However, the details thereof have not yet been clarified.

In recent years, extracorporeal blood circulation therapy is being used for the purpose of treating various diseases. For example, what is called leukocytapheresis for reducing leukocytes existing in the blood of a patient is being used for the treatment of inflammatory bowel diseases or hepatitis C virus (HCV) infection (EP Patent Application No. 319961; Japanese Patent Application Laid-Open No. 2003-235959). These treatment methods are intended to treat diseases by eliminating lymphocytes, granulocytes, or activated leukocytes from the peripheral blood.

With regard to extracorporeal circulation therapy, a phenomenon whereby the number of leukocytes contained in the peripheral blood is reduced during the circulation and the thus reduced number temporarily returns to the initial number due to overshooting after completion of the circulation has been known (Therapeutic Apheresis 6(6): 402-412, 2002). An attempt to collect specific cells that are temporarily mobilized into the peripheral blood by such an overshooting phenomenon, so as to utilize the cells for the treatment or basic research of various diseases, has also been made (Japanese Patent Application Laid-Open No. 2003-9851). However, this attempt is only directed towards the collection of hematopoietic stem cells.

The Journal of Gastroenterology, the Japanese Society of Gastroenterology, Vol. 100, No. 12, pp. 1369-1374, 2003, describes the presence of bone marrow-derived intestinal epithelial cells and the repair of the mucosal epithelium with the bone marrow-derived intestinal epithelial cells. This publication discusses the possibility of the application of such bone marrow-derived cells to the repair of the gastrointestinal epithelium. However, the publication does not teach at all that bone marrow-derived cells are induced into an injured epithelium by leukocytapheresis, so as to repair the epithelium.

### DISCLOSURE OF THE INVENTION

It is an object of the present invention to provide a method for repairing injured epithelial cells, impossibility of the repair of which results in fatal consequences.

For the purpose of achieving the aforementioned object, the present inventors have carried out leukocytapheresis on a patient with injured epithelial cells, using an extracorporeal circulation-type leukocyte removing device. Thus, the inventors have intensively studied a change in the epithelial cells of the patient. As a result, the inventors have found that epithelial cells have rapidly increased after the leukocytapheresis. Thus, the present inventors have completed the present invention based on these findings.

That is to say, the present invention provides the following features of the invention:
(1) A method for inducing bone marrow-derived cells into the injured epithelial site of a patient, which comprises removing leukocytes from the blood that has been collected from the patient with injured epithelial cells, and then retransfusing the remaining blood to said patient.
(2) The method according to (1) above, wherein the blood collected from the patient with injured epithelial cells is allowed to pass through a blood treatment means having a means for removing leukocytes from the blood and the remaining blood is then retransfused to said patient.
(3) The method according to (2) above, wherein the means for removing leukocytes from the blood is at least one selected from a fiber, a porous body, a particle, a film, a flat membrane, a hollow fiber, and a tubular substance.
(4) The method according to (1) above, wherein collection of the blood from the patient with injured epithelial cells, removal of leukocytes from said blood, and retransfusion of the remaining blood to said patient are continuously carried out by extracorporeal circulation.
(5) The method according to (1) above, wherein the injured epithelial cells are intestinal epithelial cells.
(6) The method according to (1) above, wherein the bone marrow-derived cells that are to be induced into the injured epithelial site are at least one of any of Paneth cells, goblet cells, and enteroendcrine cells.
(7) A method for repairing an injured epithelium, which comprises removing leukocytes from the blood that has been collected from a patient with injured epithelial cells, and then retransfusing the remaining blood to said patient, so as to induce bone marrow-derived cells into the injured epithelial site of said patient.
(8) The method according to (7) above, wherein the blood collected from the patient with injured epithelial cells is allowed to pass through a blood treatment means having a means for removing leukocytes from the blood and the remaining blood is then retransfused to said patient.
(9) The method according to (7) above, wherein the means for removing leukocytes from the blood is at least one selected from a fiber, a porous body, a particle, a film, a flat membrane, a hollow fiber, and a tubular substance.
(10) The method according to (7) above, wherein collection of the blood from the patient with injured epithelial cells, removal of leukocytes from said blood, and retransfusion of the remaining blood to said patient are continuously carried out by extracorporeal circulation.
(11) The method according to (7) above, wherein the injured epithelial cells are intestinal epithelial cells.
(12) The method according to (7) above, wherein the bone marrow-derived cells that are to be induced into the injured epithelial site are at least one of any of Paneth cells, goblet cells, and enteroendcrine cells.
(13) The method according to (7) above, which further comprises allowing the bone marrow-derived cells that have been induced into the injured epithelial site to differentiate into epithelial cells.
(14) The method according to (13) above, wherein the injured epithelium is repaired with the differentiated epithelial cells.
(15) The method according to (7) above, which is used in combination with a pharmacotherapy that is used for the repair of the injured epithelium.
(16) A method for treating ulcerative colitis, which comprises carrying out the method for repairing an injured epithelium according to (7) above.

### BRIEF DESCRIPTION OF THE DRAWINGS

FIG. 1 shows an example of the extracorporeal circulation system used for the method of the present invention. A blood treatment means having a means for removing leukocytes is provided at the midpoint of an extracorporeal circulation circuit. The blood flow rate may be set at 30 to 50 ml/minute, and the amount of blood treated may be set at 2 to 3 L/once, for example. The removal of leukocytes using this system is defined as leukocytapheresis (hereinafter referred to as LCAP).

FIG. 2 shows one case of injured epithelial cells observed before and after the treatment according to the method of the present invention. Three figures at the upper case show: Paneth cells identified by HE staining (left figure); goblet cells identified by Alcian Blue staining (center figure); and enteroendcrine cells identified by Chromogranin A staining (right figure), respectively, all of which were observed before subjecting to LCAP (preLCAP). Three figures at the lower case show: Paneth cells identified by HE staining (left figure); goblet cells identified by Alcian Blue staining (center figure); and gut endocrine cells identified by Chromogranin A staining (right figure), respectively, all of which were observed 8 days after completion of LCAP (postLCAP (Day 8)).

FIG. 3 shows another case of injured epithelial cells observed before and after the treatment according to the method of the present invention. Three figures at the upper case show: Paneth cells identified by HE staining (left figure); goblet cells identified by Alcian Blue staining (center figure); and enteroendcrine cells identified by Chromogranin A staining (right figure), respectively, all of which were observed before subjecting to LCAP (preLCAP). Three figures at the lower case show: Paneth cells identified by HE staining (left figure); goblet cells identified by Alcian Blue staining (center figure); and enteroendcrine cells identified by Chromogranin A staining (right figure), respectively, all of which were observed 11 days after completion of LCAP (postLCAP (Day 11)).

### BEST MODE FOR CARRYING OUT THE INVENTION

The present invention will be described in detail below.

Epithelial cells are cells that cover free surfaces existing inside or outside a body. That is, these are cells covering the surfaces of the bowel, oral cavity, esophagus, bronchus, lung, peritoneum, or the like. In the case of the bowel for example, a monolayer epithelial cell group exists on the surface of the intestinal tract and covers it. Specifically, such an epithelial cell group consists of epithelial stem cells that repeatedly self-proliferation, epithelial precursor cells existing in a proliferation zone, and main 4 types of mature epithelial cells constituting many parts of the intestinal epithelium, that is, enterocytes, goblet cells, enteroendcrine cells, and Paneth cells.

A patient with injured epithelial cells is in a state where the repair of the epithelium in the bowel, oral cavity, esophagus, bronchus, lung, peritoneum, or the like, cannot be carried out due to inflammation or ulceration. Specifically, such a state is observed in patients who suffer from ulcerative colitis, Crohn's disease, radiation enteritis, intestinal Behect's disease (simple ulcer), graft versus host disease (GVHD) enteritis, nonspecific small intestinal ulcer, stomatitis, esophagitis, bronchitis, pneumonia, peritonitis, or the like.

The term “blood” is used in the present invention to include various types of blood corpuscles, blood components such as plasma or serum, and/or a mixture thereof.

In the present invention, a specific example of the means for collecting the blood from a patient is a blood-collecting needle.

The term “means for sending the blood” is used in the present invention to specifically mean a means for transporting the blood filled in a tube. A specific example is a blood tube pump.

In the present invention, a means for retransfusing the blood to a patient specifically comprises a blood pump, a blood retransfusion needle, and the like.

In the present invention, examples of a means for removing leukocytes from the blood include: fibers such as a nonwoven fabric or woven fabric, porous bodies, granular or bead particles, films, flat membranes, hollow fibers, and tubular materials, which are all used as leukocyte removing materials. Of these, a porous body and a nonwoven fabric are preferable in that these materials have a large surface area capable of removing leukocytes. A nonwoven fabric is most preferable.

The material of such a leukocyte removing material is not particularly limited. Examples of such a material include: natural polymers such as cellulose; polyesters such as polyethylene terephthalate or polybutylene terephthalate; polyolefins such as polyethylene or polypropylene; and polymeric materials such as polyvinylidene fluoride, polyamide, polyimide, polyurethane, polysulfone, or polyacrylonitrile. The surface of these materials may further be subjected to a surface treatment, so as to improve ability to remove leukocytes or prevent the adhesion of other blood components such as platelets.

When the leukocyte removing material is a nonwoven fabric, a filament thereof may be either a monofilament or a multifilament. In addition, such a nonwoven fabric preferably has a mean fiber diameter from 0.8 μm to 40 μm. As the fiber diameter increases, it becomes difficult to maintain the surface area of a substrate, and the ability to remove leukocytes thereby decreases. Thus, too large fiber diameter is not preferable. In contrast, as a fiber diameter decreases, clogging is generated on the removing material. Thus, too small fiber diameter is not preferable either.

A cellulose acetate carrier having a bead form may also be a preferred example of the aforementioned leukocyte removing material.

A repairing device for carrying out the method of the present invention comprises at least the aforementioned blood collecting means, blood sending means, blood treating means, and blood retransfusing means. These means are liquid-tightly connected with one another in the above order via a tube for supplying the blood to be treated.

Moreover, the aforementioned repairing device may also comprise a means for adding to the blood, agents or an anticoagulant used for anticoagulation of the blood. The type of an anticoagulant is not particularly limited as long as it is a compound having anticoagulant activity. Preferred examples of such an anticoagulant include heparin, low molecular weight heparin, nafamostat mesilate, gabexate mesilate, argatroban, and sodium citrate. Of these, heparin or nafamostat mesilate are more preferable.

Furthermore, the aforementioned repairing device may further comprise a means for measuring the flow rate or pressure of the blood.

Conditions for treating the blood using the aforementioned repairing device are different depending on the target patient, symptoms of the disease of the target patient, and the like. In general, the blood may be circulated at a flow rate of 20 to 50 ml/minute for 30 minutes to 2 hours for a single operation. Naturally, such conditions may be determined as appropriate, taking into consideration the amount of a leukocyte removing material applied, the adsorptive properties thereof, etc.

The present invention enables the repair of an injured epithelium. Thus, it is also possible to use the device of the present invention in combination with various types of treatment methods for repairing such an injured epithelium, such as a pharmacotherapy, before, after, or both before and after the treatment with the device of the present invention.

The present invention will be described further in detail in the following examples. However, the examples are not intended to limit the scope of the present invention.

### EXAMPLES

Leukocytapheresis was carried out on 8 patients suffering from ulcerative colitis under the conditions indicated below.

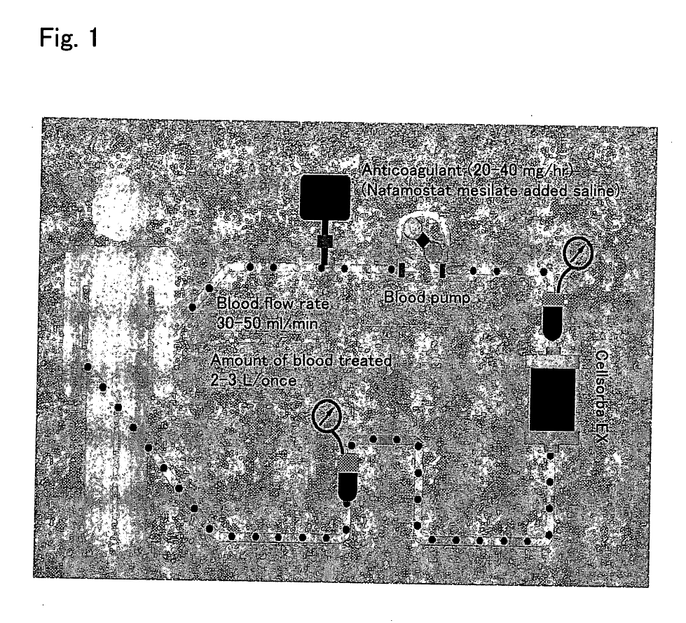

A system configuration of extracorporeal circulation is shown in FIG. 1.

As a blood treating means, Cellsorba (registered trademark) EX (manufactured by Asahi Kasei Medical Co., Ltd.; an adsorptive blood depurator, the internal portion of which is filled with a cylindrical polyester nonwoven fabric).

As shown in FIG. 1, using a blood pump, the blood was collected from the cubital fossa vein or femoral vein and then introduced into a circulation circuit. The blood was then supplied into Cellsorba EX. The treated blood was passed through the circuit and then retransfused to the other cubital fossa vein or femoral vein that was located on the side opposite to the blood collection side. The blood flow rate was 30 to 50 ml/minute, and 2 to 3 L of the blood was treated in a single operation. In addition, in order to prevent blood coagulation in the circuit, nafamostat mesilate was injected in the circuit.

The effect of repairing injured epithelial cells was confirmed by the following method.

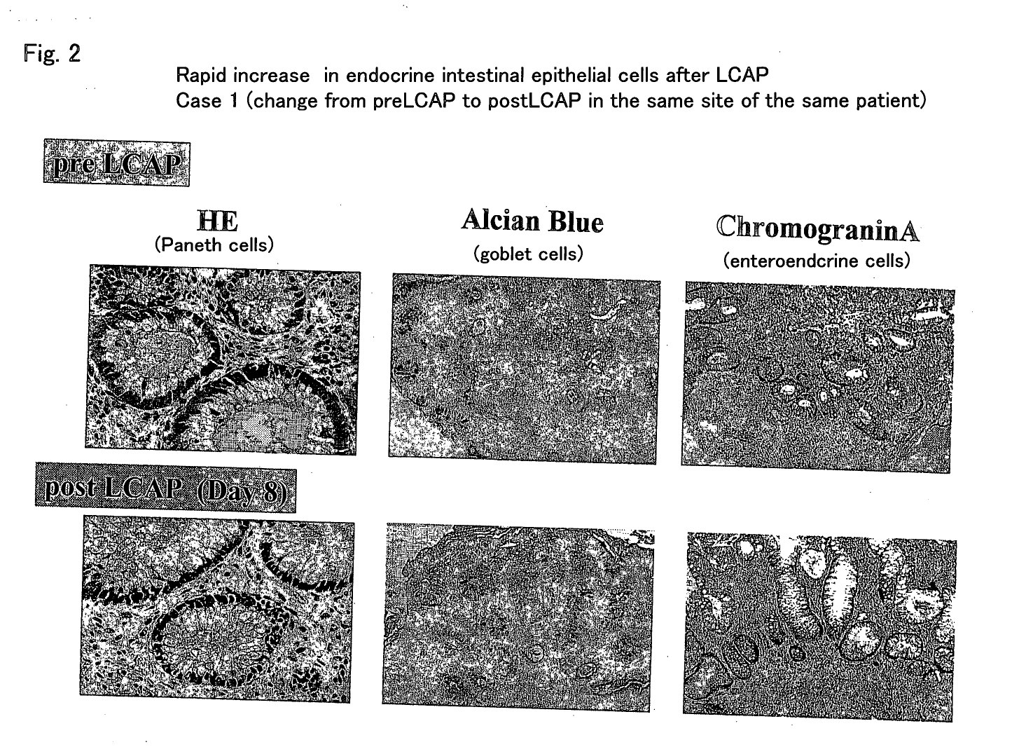

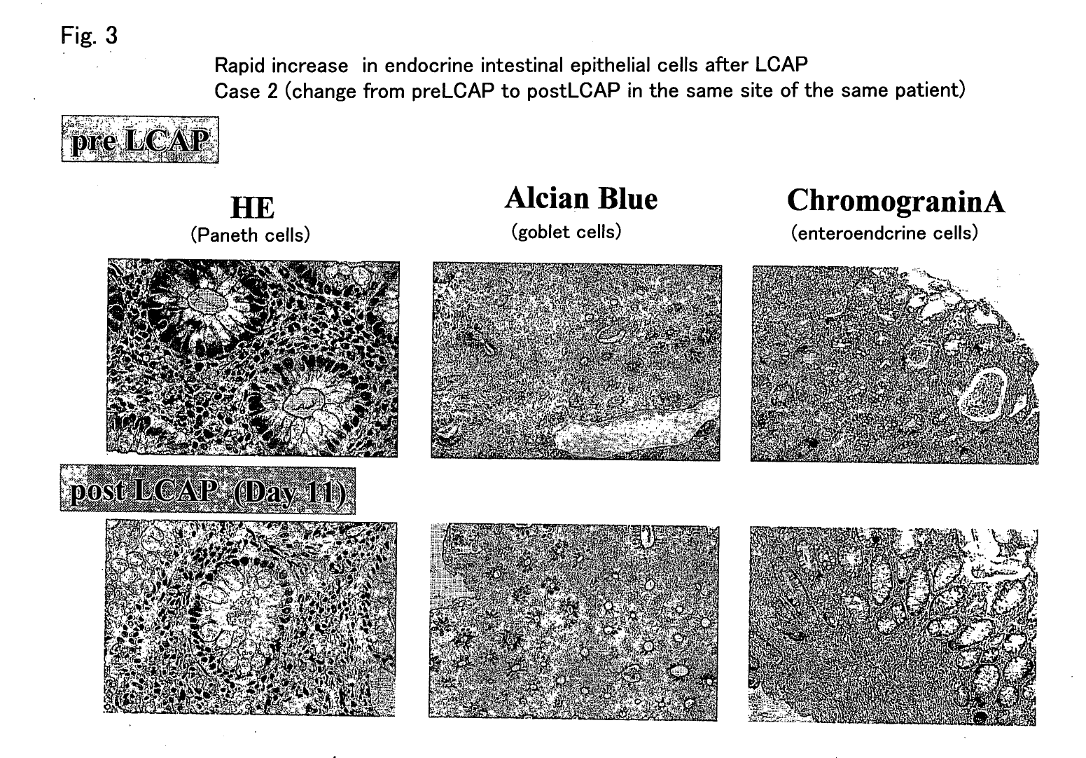

Colonoscopy was carried out on the day before the leukocytapheresis and 8 to 11 days thereafter (after completion of the second operation thereof), so as to observe the inside of the bowel with an endoscope. A significant difference was not observed among the cases. Thereafter, biopsy tissues were collected from the same portion of the rectum of each patient under observation with an endoscope, and a change over time in mature epithelial cells in the same case was observed before and after the leukocytapheresis. Consequently, it was found that Paneth cells identified by HE staining, goblet cells identified by Alcian Blue staining, and enteroendcrine cells identified by Chromogranin A staining rapidly increased, and that as a result, the symptoms of 5 patients were improved. Leukocytapheresis gave therapeutic effects on the 5 patients having a rapid increase in mature endocrine intestinal epithelial cells, more significantly and at an earlier stage, than on the remaining 3 patients that had not exhibit such a rapid increase. The results of 2 cases out of the above 5 cases, in which a rapid increase in epithelial cells was observed, are shown in FIGS. 2 and 3. FIGS. 2 and 3 show the photographs of the stained Paneth cells, goblet cells, and enteroendcrine cells that were taken at the same site of a single patient. Photographs at the upper case were taken before the leukocytapheresis. On the other hand, photographs at the lower case were taken 8 days and 11 days after the leukocytapheresis, respectively.

As is clear from the above-described results, leukocytapheresis enables induction of epithelial cells, thereby exhibiting the effect of repairing an injured epithelium. Based on previous findings (please refer to, for example, the aforementioned Journal of Gastroenterology, the Japanese Society of Gastroenterology, Vol. 100, No. 12, pp. 1369-1374, 2003), it is considered that the thus induced epithelial cells are derived from the bone marrow. Moreover, it is considered that a therapeutic effect on intractable inflammatory bowel diseases is exhibited through the repair of an injured epithelium.

Effect of the Invention

The present invention provides a method for repairing injured epithelial cells, impossibility of the repair of which results in fatal consequences. It is anticipated that the application of the method of the present invention enables the treatment of various diseases associated with injured epithelial cells.

1. A method for inducing bone marrow-derived cells into the injured epithelial site of a patient, which comprises removing leukocytes from the blood that has been collected from the patient with injured epithelial cells, and then retransfusing the remaining blood to said patient.

2. The method according to claim 1, wherein the blood collected from the patient with injured epithelial cells is allowed to pass through a blood treatment means having a means for removing leukocytes from the blood and the remaining blood is then retransfused to said patient.

3. The method according to claim 2, wherein the means for removing leukocytes from the blood is at least one selected from a fiber, a porous body, a particle, a film, a flat membrane, a hollow fiber, and a tubular substance.

4. The method according to claim 1, wherein collection of the blood from the patient with injured epithelial cells, removal of leukocytes from said blood, and retransfusion of the remaining blood to said patient are continuously carried out by extracorporeal circulation.

5. The method according to claim 1, wherein the injured epithelial cells are intestinal epithelial cells.

6. The method according to claim 1, wherein the bone marrow-derived cells that are to be induced into the injured epithelial site are at least one of any of Paneth cells, goblet cells, and enteroendcrine cells.

7. A method for repairing an injured epithelium, which comprises removing leukocytes from the blood that has been collected from a patient with injured epithelial cells, and then retransfusing the remaining blood to said patient, so as to induce bone marrow-derived cells into the injured epithelial site of said patient.

8. The method according to claim 7, wherein the blood collected from the patient with injured epithelial cells is allowed to pass through a blood treatment means having a means for removing leukocytes from the blood and the remaining blood is then retransfused to said patient.

9. The method according to claim 7, wherein the means for removing leukocytes from the blood is at least one selected from a fiber, a porous body, a particle, a film, a flat membrane, a hollow fiber, and a tubular substance.

10. The method according to claim 7, wherein collection of the blood from the patient with injured epithelial cells, removal of leukocytes from said blood, and retransfusion of the remaining blood to said patient are continuously carried out by extracorporeal circulation.

11. The method according to claim 7, wherein the injured epithelial cells are intestinal epithelial cells.

12. The method according to claim 7, wherein the bone marrow-derived cells that are to be induced into the injured epithelial site are at least one of any of Paneth cells, goblet cells, and enteroendcrine cells.

13. The method according to claim 7, which further comprises allowing the bone marrow-derived cells that have been induced into the injured epithelial site to differentiate into epithelial cells.

14. The method according to claim 13, wherein the injured epithelium is repaired with the differentiated epithelial cells.

15. The method according to claim 7, which is used in combination with a pharmacotherapy that is used for the repair of the injured epithelium.

16. A method for treating ulcerative colitis, which comprises carrying out the method for repairing an injured epithelium according to claim 7.
